(19) United States
(12) Patent Application Publication
(10) Pub. No.: US 2019/0223788 A1
(43) Pub. Date: Jul. 25, 2019

(54) Determination of Body Fat Content by Body-Volume-Distribution and Body-Impedance-Measurement

(71) Applicant: Naked Labs Austria GMBH, Wien (AT)

(72) Inventors: Gerhard Schultes, Wien (AT); Peter Kreuzgruber, Wien (AT)

(73) Assignee: Naked Labs Austria GMBH, Wein (AT)

(21) Appl. No.: 16/317,190

(22) PCT Filed: Jul. 13, 2017
(86) PCT No.: PCT/EP2017/067668

§ 371 Date: Jan. 11, 2019

(30) Foreign Application Priority Data
Jul. 13, 2016 (DE) 10 2016 112 899.6

(51) Int. Cl. A61B 5/00; A61B 5/053; A61B 5/107; G06T 17/00; G01G 19/50; G16H 50/30

(52) U.S. Cl. CPC A61B 5/4872; A61B 5/0537; A61B 5/0064; A61B 5/1073; G16H 50/30; G06T 17/00; A61B 5/0077; G01G 19/50; A61B 5/1079

(57) ABSTRACT

A method for calculating the body fat content of a human includes the steps of measuring the body's mass and bio-electrical impedance. A 3D body scanner is used to create a digital 3D body model from which a body volume geometry is determined as a plurality of segments in a cylindrical or conical form with a measured length and a measured cross-section at each end of each segment. Taking account of the body volume geometry, each segment is assigned an electrical impedance to form an electrical body model. Taking account of the mass and electrical impedance of each segment, a body fat content of the segment is calculated. A body composition model can be calculated by summing the body fat contents of the segments of the body. A 3D body scanner for calculating the body fat content of a human is also disclosed.

### CROSS-REFERENCE TO RELATED APPLICATIONS

The present application is related by subject matter to the following concurrently filed PCT applications (all of which designate the US):

a. International Application No.: PCT/EP2017/067668; entitled “Determination of Body Fat Content by Body-Volume-Distribution and Body-Impedance-Measurement,” which claims priority to German Application No.: DE10 2016 112 899.6.

b. International Application No.: PCT/EP2017/067669; entitled “Optical Marker to Adjust the Turntable of a 3D Body Scanner”.

c. International Application No.: PCT/EP2017/067761; entitled “Efficient Volumetric Reconstruction with Depth Sensors”.

d. International Application No.: PCT/2017/067672; entitled “Skeleton Estimation from Body Mesh”.

e. International Application No.: PCT/2017/067667; entitled “Method for Creating a 3D-Model and 3D-Body-Scanner”.

f. International Application No.: PCT/2017/067664; entitled “Smart Body Analyzer with 3D Body Scanner and Vital Parameter Sensors”,

g. International Application No.: PCT/EP2017/067665; entitled “Motor Driven Turntable with Foldable Sensor Mast,” which claims priority to German Application No.: DE 10 2016 112 893.7.

h. International Application No.: PCT/EP2017/067671; entitled “Alignment of Scan Parts on a Turntable,” which claims priority to German Application No.: DE 10 2016 112 890.2.

The above cited PCT international applications are hereby incorporated herein in their entireties by this reference for all purposes. Any combination of the features and aspects of the subject matter described in at least one of the incorporated applications may be combined with embodiments of the present application to yield still further embodiments of the present invention.

### FIELD OF THE INVENTION

The invention relates to a method for calculating the body fat content of a human. Further, the invention relates to a 3D body scanner for calculating the body fat content of a human.

### BACKGROUND OF THE INVENTION

Body composition in fat tissue, muscular tissue and others is demanded by various fields of applications such as: Fitness, Body styling, Medical applications and/or Health care applications.

To determine body composition, various methods in a wide range of cost and accuracy are used.

Simple Calculation from BMI:

Body fat can be estimated from Body Mass Index (BMI), which is a person's mass in kilograms divided by the square of the height in meters. There are a number of proposed formulas that relate body fat to BMI. These formulas are based on work by researchers published in journals, but their correlation with body fat is only estimated. Body fat of any particular individual cannot be deduced accurately from BMI.

Height and Circumference Methods:

The U.S. Navy circumference method compares abdomen or waist and hips measurements to neck measurement and height and other sites to claim to estimate one's body fat percentage by a conversion from the BMI. There is limited information, however, on the validity of the so-called “rope and choke” method because of its universal acceptance as inaccurate and easily falsified. The U.S. Army and U.S. Marine Corps also rely on the height and circumference method. For males, they measure the neck and waist just above the navel. Females are measured around the hips, waist, and neck. These measurements are then looked up in published tables, with the individual's height as an additional parameter. This method is used because it is a cheap and convenient way to implement a body fat test throughout an entire service. Methods using circumference have little acceptance outside the Department of Defense due to their negative reputation in comparison to other methods.

Skin-Fold Method:

At the skin-fold estimation method, also known as a pinch test, a pinch of skin is measured by calipers at several standardized points on the body to determine the subcutaneous fat layer thickness. These measurements are converted to an estimated body fat percentage by an equation. Some formulas require as few as three measurements, others as many as seven. The accuracy of these estimates is more dependent on a person's unique body fat distribution than on the number of sites measured. As well, it is of utmost importance to test in a precise location with a fixed pressure. Skin-fold-based body fat estimation is also sensitive to the type of caliper used, and technique. This method also measures only subcutaneous adipose tissue (fat under the skin). Two individuals might have nearly identical measurements at all of the skin fold sites, yet differ greatly in their body fat levels due to differences in other body fat deposits such as visceral adipose tissue: fat in the abdominal cavity. Although it may not give an accurate reading of real body fat percentage, it is a reliable tool to track body composition change over a period of time, provided tile test is carried out by the same person with the same technique.

Ultrasonic Method:

Ultrasound has proven to be an accurate technique to measure subcutaneous fat thickness. Ultrasound systems rely on using tabulated values of tissue sound speed and automated signal analysis to determine fat thickness. By making thickness measurements at multiple sites on the body, the body fat percentage can be calculated. Ultrasonic techniques can also be used to directly measure muscle thickness and quantify intramuscular fat. Ultrasonic equipment is expensive, and thus is not cost-effective for body fat measurement. A weakness of ultrasonic measurements is the conclusion from a limited number of measurements to the full body without precise information of the body shape.

Underwater Weighing:

Fat cells in humans have an average density of about 0.9 kilograms per liter and a value of 1.1 kilograms per liter for the density of the “fat free mass”. With a weighing system, body density can be determined with great accuracy by completely submerging a person in water and calculating the volume from the weight of the displaced water. A correction is made for the buoyancy of air in the lungs and other gases in the body spaces. An uncertainty in fat estimation would be about ±3.8% of the body weight, because of normal variability in body constituents.

Whole-Body Air Displacement Plethysmography:

Whole-body air displacement plethysmography (ADP) is a recognized and scientifically validated densitometric method to measure human body fat percentage. ADP uses the same principles as the underwater weighing, but representing a densitometric method that is based on air displacement rather than on water immersion. Air-displacement plethysmography offers several advantages over established reference methods, including a quick, comfortable, automated, noninvasive, and safe measurement process, and accommodation of various subject types (e.g., children, obese, elderly and disabled persons). However, because the basic body constitution of the subject is unknown, the accuracy of the ADP method declines at the extremes of body fat percentages by 1.68-2.94% and tends to overstate percent body fat in very lean subjects with up to a 13% error.

Near-Infrared Interactance:

A beam of infra-red light is transmitted into a region of the subject's body, e.g. the biceps. The light is reflected from the underlying muscle and absorbed by the fat. The method is safe, noninvasive, rapid and easy to use, however not fully validated and not frequently used until now.

Dual Energy X-Ray Absorptiometry:

Dual energy X-ray Absorptiometry, (DXA) is a newer method for estimating body fat percentage, and determining body composition and bone mineral density. X-rays of two different energies are used to scan the body, one of which is absorbed more strongly by fat than the other. A computer can subtract one image from the other, and the difference indicates the amount of fat relative to other tissues at each point. A sum over the entire image enables calculation of the overall body composition. DXA is expensive, and exposes the body significantly to X-ray so that it is not recommended for everyday use but just for reference or calibration measurement for other methods.

Further Complicated Methods:

There are several more complicated procedures that more accurately determine body fat percentage. Some, referred to as multicompartment models, can include DXA measurement of bone, plus independent measures of body water (using the dilution principle with isotopically labeled water) and body volume (either by water displacement or airplethysmography). Various other components may be independently measured, such as total body potassium. In-vivo neutron activation can quantify all the elements of the body and use mathematical relations among the measured elements in the different components of the body (fat, water, protein, etc.) to develop simultaneous equations to estimate total body composition, including body fat.

### Brief Objects and Summary of the Invention

The object of the present invention is to provide both a method for calculating the body fat content of a human and a 3D body scanner for calculating the body fat content of a human, which eliminates the disadvantages of the prior art.

The aforementioned object is achieved by means of a method for calculating the body fat content of a human and of a 3D body scanner for calculating the body fat content of a human exhibiting the features disclosed in the description that follows.

Proposed is a method for calculating the body fat content of a human with the following steps: measuring the bio-electrical impedance of a human body; measuring the mass of the human body; creating with a 3D body scanner a digital 3D body model of the human body; determine a body volume geometry from the digital 3D body model, wherein the body volume geometry comprises a plurality of segments in a cylindrical and/or conical form with a measured length and a measured cross-section at each end; creating from the body volume geometry and the bio-electrical impedance of the body an electrical body model with, in particular detailed, segmental impedances for each segment; calculating, in particular in consideration of the mass, for each segment with the segmental impedance a part-body fat content; and calculating a body composition model by summing the part-body fat contents of the segments up to the full body.

It is advantageous if the segmental impedance of each segment is calculated in consideration of the bio-electrical impedance of the body and the body volume geometry, in particular of the measured length and cross-sections of each segment. Thus, the volume and the calculated segmental impedance of each segment are known.

It is advantageous if the segments have a natural form,

In an advantageous further aspect, at least one, in particular really by a 3D scanner measured, segment for each limb and the trunk of the body is introduced.

It is advantageous if a plurality of segments is introduced for the trunk and/or for each limb, in particular for the shoulder joint, the upper arm, the forearm, the hand, the thigh, the knee joint and/or the lower leg.

It is advantageous if the mass of the human body is measured with a build-in scale of the 3D body scanner.

In an advantageous further aspect, the bio-electrical impedance of the human body is measured with at least two electrodes of the 3D body scanner, in particular of the build-in scale.

It is advantageous if the height of the human body is measured, in particular from the body volume geometry, and/or if the body volume geometry comprises the height of the human body.

In an advantageous further aspect, the body constitution and/or the sex of the human is obtained from the digital 3D body model and/or is considered when calculating the body composition model.

It is advantageous if the measurement of the bio-electrical impedance, the measurement of the mass, height and/or the scanning of body geometry are performed at the same time.

Proposed is a 3D body scanner for calculating the body fat content of a human comprising: at least two electrodes for measuring a bio-electrical impedance of the human body; a scale for measuring a mass of the human body; a scanner for scanning the human body; and a processor for creating a digital 3D body model, which is designed to operate with a method for calculating the body fat content of a human as set forth in one or several of the previous claims.

It is advantageous if at least two electrodes and/or the build-in scale are integrated into a turntable of the 3D body scanner for rotating the human body around a turntable rotation axis.

In an advantageous further aspect, the scanner comprises at least one stationary camera for scanning the human body.

For bio electrical impedance analysis (BIA) method it is advantageous if two or more electrodes are attached to a person's body and a small AC current is sent through the body whereas the voltage between the electrodes is measured and the complex impedance Z can be determined. The, in particular complex, impedance between the conductors allows an estimate of body fat between the electrodes, because the impedance varies between fat, muscular and skeletal tissue. Fat-free muscle mass and blood tubes are a good conductor as it contains a large amount of water and electrolytes, while fat and air in e.g. the lounges are anhydrous and a poor conductor of electric current.

It is advantageous if in practical measurement, each bare foot is placed on an electrode, where the current is sent up one leg, across the abdomen and down the other leg. Additionally or alternatively, an electrode may be held in each hand; calculation of fat percentage uses the weight, so that must be measured with scales and entered by the user. The two methods may give different percentages, without being inconsistent, as they measure fat in different parts of the body. More sophisticated instruments use electrodes for both feet and hands.

In contrast to state of the art instruments the method uses the full information about the body geometry available and calculates with significantly improved accuracy the distribution of impedances between the body parts.

These measurements result in a simple electrical body model of complex impedances Z. The complex impedances Z are representing the complex resistance of the limbs and the trunk. From this electrical model the body composition, especially the fat content can be calculated.

The measurement works well under the assumption that length and the cross-section of the limbs and the trunk, being involved in the current path of the measurement, are known. However, they are typically unknown by the BIA analyzer. The measured body impedance is significantly influenced by the length and cross-section of the limbs and the trunk which is unknown by the BIA analyzers. Generally, BIA is a low cost and low space consuming method, which makes it popular in private and medical use.

It is advantageous if a 3D Body Scanner is used. The 3D body scanner creates a digital 3D body model of the human to be scanned. The achieved accuracy is in mm range. From this significant body data as height, volume length, circumference and cross-section of limbs, trunk and head can be derived. Usually also mass is provided by a built-in scale. Due shading and hair to typically not 100% of the body surface are “seen” by the scanner. Therefore, some assumptions of the missing body contours have to be made, introducing some individual dependent error.

It is advantageous if the mathematics to determine body composition from mass and volume is comparable or equal to that of underwater weighting or ADP but can be extended by more body geometry information to improve performance at very fat or slim subjects.

3D body scanning and bio-electrical impedance analysis (BIA) are the most popular low cost methods to determinate body composition from fat and non-fat tissue. 3D body scanning provides a digital 3D body model, has the information about body constitution but the inner composition of the body is unknown. BIA measures indirectly the body composition by body impedance measurement. We propose that combining both methods can in an advantageous way generate a full set of geometrical and inner body tissue distribution information. This overcomes the weaknesses of both methods and gives highly accurate results at low cost of apparatus.

### BRIEF DESCRIPTION OF THE DRAWINGS

Additional advantages of the invention are described in the following exemplary embodiments. The drawings show in:

FIG. 1 an electrical body model composed by concentrated complex impedances of the limbs and the trunk and

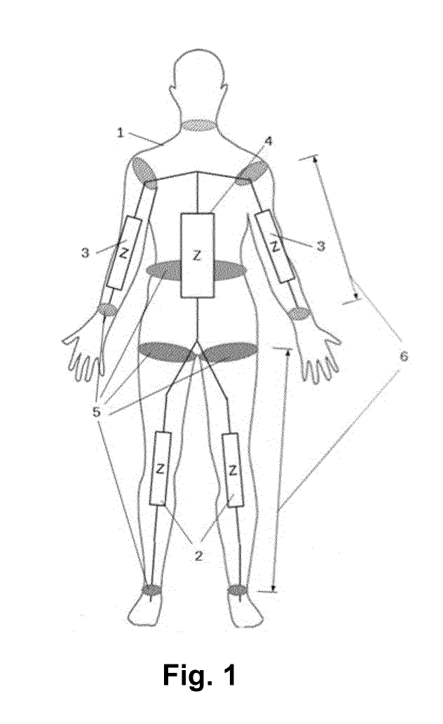

FIG. 2 an electrical body model composed by more distributed complex impedances modeling the limbs and the trunk.

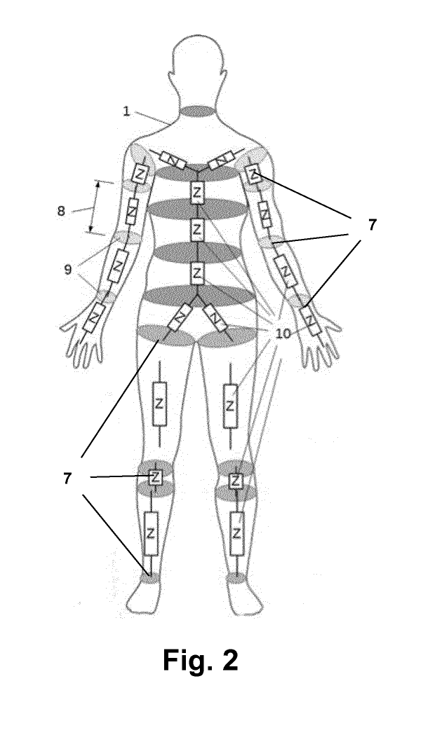

FIG. 3 a schematic representation of a 3D body scanner and components thereof.

### DETAILED DESCRIPTION OF EXEMPLARY EMBODIMENTS OF THE INVENTION

The field of the disclosed invention deals with combining the two most popular and low cost methods, which are 3D body scanning and bio-electrical impedance analysis, while seeking to reduce the weaknesses of both methods.

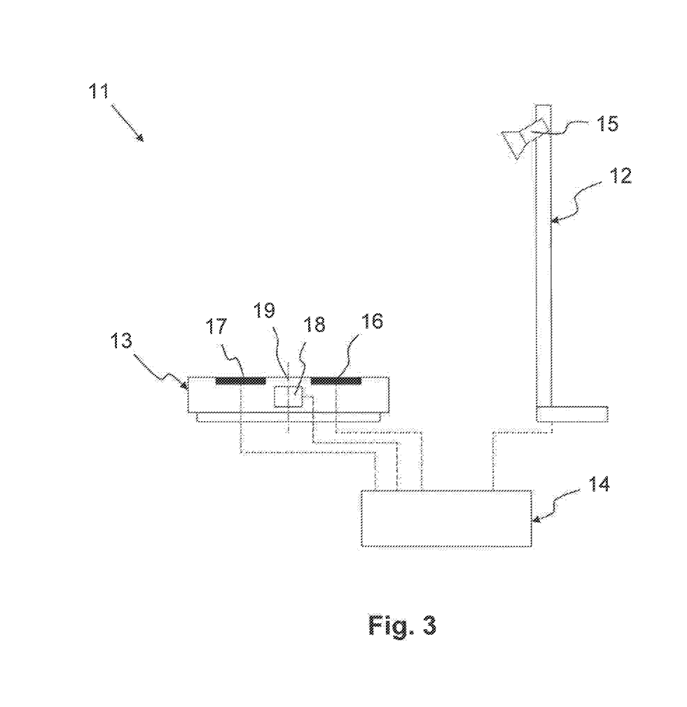

As schematically shown in FIG. 3, 3D body scanners can be employed to derive body composition as e.g. body fat content from mass and volume and with object analysis considering the sex of the scanned body and using knowledge about specific mass of fat and non-fat tissue. Weakness in this method is the accuracy of volume determination by invisible body parts and the effects of motion during the scanning process. As schematically shown in FIG. 3, the 3D body scanner includes a scanner 12, a turntable 13, a processor 14, a camera 15, a first electrode 16, a second electrode 17 and a scale 18. The turntable 13 is rotatable in front of the scanner 12 about a turntable axis 19.

Bio-electrical impedance analysis (BIA) determines the inner composition of the body 1 by the means of the complex impedance of the body. Knowing the specific resistance of fat and non-fat tissues of the body, a body composition model, especially giving fat and muscular content, is calculated,

Practically this is done in BIA by measuring the electrical complex sum impedances between the various limbs. Therefrom an electrical body model consisting of integral values of the complex impedances of the legs 2, arms 3, and the trunk 4 is created.

The dependency of these impedances on the cross-section 5 and length 6 of the limbs and the trunk is considered by formulas that make corrections based on statistical data from the population about the body geometry. This also explains significant measurement errors dependent on the ethnic group and the body dimension of the tested individual.

In accordance with the present invention, the body volume geometry and mass data of an individual obtained by 3D body scanning technology is combined with the information of BIA about the inner composition of the body from impedance analysis to overcome the weaknesses inherent in both methods.

The 3D body model enables the generation of an electrical body model with a much lower granulation by introducing a higher number of limb and trunk segments in a cylindrical or conical form with individual length 8 and cross section 9 which may have a natural form. For each segment with a segmental impedance 10, an individual part-body fat content can be calculated and summed up to the full body. The ends of at least one segment can be positioned in the region of two adjacent joints 7 of the body 1.

In other words: By combining 3D body scanning technology with BIA, the weakness of volume determination and motion during the scanning process of 3D body scanning can be overcome by BIA. At the same time, the weakness of BIA of unknown body geometry is eliminated by body constitution information provided by 3D body scanning technology.

It is proposed to calculate the body fat content of a human by combining real geometrical information such as (but not limited to) measured body height, measured limbs length 6 and/or cross-section 5 and/or measured trunk length 6 and/or cross-section 5, with the electrical measurements of BIA to obtain in an advantageous way the body fat content independent of the experimentee's age, basic body constitution, ethnic affiliation and/or possible handicap and even under circumstances of the experimentee's extreme slimness or obesity.

It is proposed to calculate the body fat content of a human by combining detailed geometrical information such as (but not limited to) measured body height, measured limbs segment length 8 and/or segment cross-section 9, measured trunk segment length 8 and/or segment cross-section 9, at high granularity with the electrical measurements of BIA to obtain in an advantageous way the body fat content independent of the person's age, basic body constitution, ethnic affiliation and/or possible handicap and even under circumstances of the person's extreme slimness or obesity.

Mass and geometrical like height and/or volume of the person may in an advantageous way automatically be obtained from 3D body measured scanner data using a 3D body scanner 11 as schematically shown in FIG. 3. Body constitution and/or sex of the person may be in an advantageous way automatically obtained from the 3D body scanner measured data. 3D body scanning and BIA is combined in an advantageous way in one apparatus. 3D body scanning and BIA is performed in an advantageous way at the same time. 3D body scanning and BIA in an advantageous way uses the same body pose.

The invention is not limited to the embodiments shown or described. Rather, any and all combinations of the individual features described, as shown in the figures or described in the description, and to the extent that a corresponding combination appears possible and sensible, are subject matters of the invention.

3D body scanning and bio-electrical impedance analysis (BIA) are the most popular low cost methods to determinate body composition from fat and non-fat tissue. 3D body scanning suffers from low precision of volume determination and unknown inner composition of the body. BIA suffers from not knowing the body's volume and the geometrical constitution of the body. Combining both methods can in an advantageous way overcome the aforementioned weaknesses and accordingly achieve highly accurate results with an apparatus that can be made available at relatively low cost.

### LIST OF REFERENCE CHARACTERS

1. Body

2. Complex impedances of the legs

3. Complex impedances of the arms

4. Complex impedances of the trunk

5. Cross-section of limbs and trunk

6. Length of limbs and trunk

7. Joint

8. Length of limb or trunk segment.

9. Cross-section of limb or trunk segment

10. High granulated complex impedances of the limbs and trunk,

11. 3D body scanner

12. Scanner.

13. Turntable.

14. Processor.

15. Camera.

16. First electrode.

17. Second electrode.

18. Scale.

19. Turntable axis of rotation.

1. Method for calculating the body fat content of a human body with the following steps:
measuring the bio-electrical impedance of the human body;
measuring the mass of the human body;
creating with a 3D body scanner a digital 3D body model of the human body;
determine a body volume geometry from the digital 3D body model, wherein the body volume geometry comprises a plurality of segments in a cylindrical and/or conical form with a measured length and a measured cross-section at each end of each segment;
creating from the body volume geometry and the bio-electrical impedance of the human body an electrical body model with segmental impedances for each segment;
taking account of the mass of each segment and the segmental impedance of each segment in calculating a part-body fat content for each segment; and
aggregating the body fat contents of theplurality of segments in calculating a body composition model for the human body.

2. Method according to claim 1, wherein the segmental impedance of each segment is calculated in consideration of the bio-electrical impedance of the body and the measured length and cross-sections of each segment.

3. Method according to claim 1, wherein the segments have a natural form.

4. Method according to claim 1, wherein each limb is defined by at least one segment and the trunk of the body is defined by at least one segment.

5. Method according to claim 1, wherein a shoulder joint is defined bv at least one segment, an upper arm is defined bv at least one segment, a forearmjs defined bv at least one segment, a hand is defined bv at least one segment, a thigh is defined bv at least one segment, a knee joint is defined bv at least one segment and a lower leg is defined by at least one segment.

6. Method according to claim 1. wherein the mass of the human body is measured with a build-in scale of the 3D body scanner.

7. Method according to claim 1. wherein the bio-electrical impedance of the human body is measured with at least two electrodes of the 3D body scanner.

8. Method according to claim 1, the body volume geometry comprises the height of the human body.

9. Method according to claim 1, wherein the sex of the human body is is considered when calculating the body composition model.

10. Method according to claim 1, wherein the measurement of the bio electrical impedance, the measurement of the mass, height and the scanning of body geometry are performed at the same time.

11. 3D body scanner for calculating the body fat content of a human, the 3D body scanner comprising:
a first electrode configured for measuring a bio-electrical impedance of the human body;
a second electrode configured foe measurincta bio-electrical impedance of the human body;
a scale for measuring a mass of the human body;
a scanner for scanning the human body; and
a processor for creating a digital 3D body model, the processor beind connected to the first electrode, the second electrode the scale and the scanner. which-Es the processor being designed to operate with a method for calculating , fat content of a human body comprisinctthe following steps:
measuring the bio-electrical impedance of the hur an body:
measuring the mass of the human body;
creating with a 3D body scanner a digital 3D body model of the human body;
determining a body volume geometry from the digital 3D body model, wherein the body volume geometry includes a plurality of segments in a cylindrical and/or conical form with a measured length and a measured cross-section t each end of each segment;
creating from the body volume geometry and the bio.-electrical impedance of the human body an electrical body model with segmental impedances for each segment;
taking account of the mass of each segment and the segmental impedance of each segment in calculating a part-body fat content for each segment; and
aggregating the body fat contents of the plurality of segments in calculating a body composition model for the human body.

12. 3D body scanner according to the claim 11, further comprising a turntable that is rotatable about a turntable axis and configured for rotating the human body about the turntable axis, wherein the two electrodes and the scale are integrated into the turntable.

13. 3D body scanner according to claim 11, wherein the scanner comprises at least one stationary camera configured and disposed for scanning the human body.
